(19) United States
(12) Plant Patent Application Publication
(10) Pub. No.: US 2011/0252529 P1
(43) Pub. Date: Oct. 13, 2011

(54) Hemerocallis plant named 'Striped Fantastic'

(50) ;

(76) Inventors: Steven Michael Brigham, Westport, CA (US); Donna Gunderson Brigham, Westport, CA (US)

(21) Appl. No.: 12/798,880

(22) Filed: Apr. 12, 2010

(51) Int. Cl. A01H 5/00

(52) U.S. Cl. PLT/263.1

(57) ABSTRACT

A new and distinct cultivar of Hemorocallis plant characterized by striking variegated leaves combined with the beautiful and large flowers of Hemorocallis ‘Kate Carpenter’.

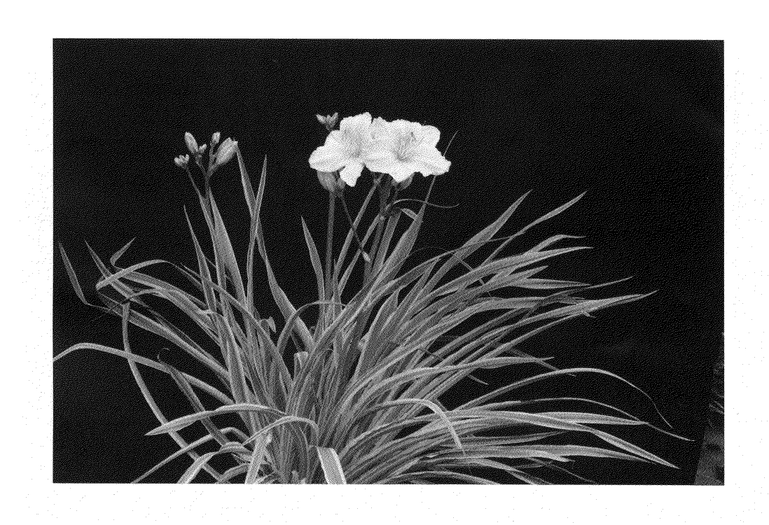

Botanical Classification: Hemorocallis hybrid

Variety Denomination: ‘Striped Fantastic’

### BACKGROUND OF THE INVENTION

The present invention relates to a new and distinct cultivar of an herbaceous perennial of the genus, Hemorocallis, and known by the cultivar name of ‘Striped Fantastic’. The genus Hemorocallis is a member of the family Hemerocallidaceae. It is natural mutation of Hemorocallis ‘Kate Carpenter’ (an unpatented plant) found in San Marcos, Calif. in the spring of 1999. Hemorocallis ‘Kate Carpenter’ was bred by R. W. Munson in 1980.

### SUMMARY OF THE INVENTION

This new cultivar is characterized by:

1) Dark green leaves with broad, creamy white margins giving the plant a long season of interest.

2) Excellent vigor.

This new cultivar has been reproduced only by asexual propagation (division and tissue culture). Each of the progeny exhibits identical characteristics to the original plant. Asexual propagation by tissue culture using standard micropropagation techniques with terminal and lateral shoots, as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWING

The drawing shows a two-year-old Hemorocallis ‘Striped Fantastic’ growing in the ground in full sun in the trial field in September in Canby, Oreg.

### DETAILED PLANT DESCRIPTION

The following is a detailed description of the new cultivar based on observations of two-year-old specimens in September grown in the ground in full sun in the trial garden in Canby, Oreg. The color descriptions are all based on The Royal Horticultural Society Colour Chart, 3^{rd} edition.
Plant:
   Type.—Herbaceous perennial.
   Form.—Arching.
   Hardiness.—USDA Zones 5 to 9.
   Size.—90 cm wide and 70 cm tall from the soil surface to the top of the foliage.
   Habit.—Clumping.
Leaf:
   Type.—Simple, basal.
   Arrangement.—Rosette, two ranked.
   Shape.—Linear, tapered, folded at the base.
   Venation.—Parallel.
   Margins.—Entire, sometimes slightly undulate.
   Apex.—Attenuate.
   Base.—Clasping.
   Size.—72 cm long and 3 cm wide.
   Surface texture.—Glabrous.
   Leaf color.—topside deep green in the leaf center, Green 137A to 138B, with a broad creamy white margin (5 to 10 mm), Yellow 4A, which narrows toward the tip and breaks into several stripes; bottom side Green 138B with creamy white margins and stripes, Yellow 4A.
Inflorescence, flower bud, and flowers are identical to the mother plant, Hemorocallis ‘Kate Carpenter’.
The description by the American Hemorocallis Society for Hemorocallis ‘Kate Carpenter’ is as follows: “The 1996 winner of the Lenington All-American Award_voted annually by the AHS Board of Directors for a daylily that performs well over a wide geographic area_is KATE CARPENTER (Munson, R. W. 1980).
KATE CARPENTER (Munson, R. W. 1980) is a stylish very round, flat and ruffled creamy melon to creamy pink self with a cream throat. Height: 28″; Flower Size: 6″; Flower Characteristics: tetraploid, diurnal, fragrant; Season of Bloom: early, midseason, reblooms; Foliage habit: evergreen. Parents: [(WILBUR HARDING×KECIA)×PAGODA GODDESS]. Awards: Junior Citation 1980, Honorable Mention 1986, Award of Merit 1989. Most Popular in AHS Region 7—1988, 1992; Lenington All-American Award 1996.”
Comparisons to similar varieties: Compared to its mother plant, Hemorocallis ‘Kate Carpenter’ (an unpatented plant), the only difference is the leaf variegation. Hemorocallis ‘STRIPED FANTASTIC’ is variegated with creamy yellow margins while Hemorocallis Kate Carpenter' is not. Compared to Hemorocallis ‘Malja’ also known as ‘Striped Zebra’ (U.S. Plant Pat. No. 14,221), the new variety is larger in size, has a lighter yellow variegation, and has larger flowers of creamy shades rather than yellow orange.

We claim:

1. A new and distinct hybrid of Hemorocallis plant substantially as shown and described.
